(19) United States
(12) Patent Application Publication
(10) Pub. No.: US 2025/0221910 A1
(43) Pub. Date: Jul. 10, 2025

(54) BODY WASH COMPOSITIONS WITH MAILLARD REACTION INHIBITING ENZYMES

(71) Applicant: HENKEL AG & CO. KGAA, Düsseldorf (DE)

(72) Inventors: Daniel Thomas PIORKOWSKI, Fairfield, CT (US); Joseph D. DAHLMEYER, JR., East Haven, CT (US)

(21) Appl. No.: 18/990,218

(22) Filed: Dec. 20, 2024

Related U.S. Application Data
(60) Provisional application No. 63/619,586, filed on Jan. 10, 2024.

(51) Int. Cl. A61K 8/66; A61K 8/41; A61K 8/60; A61Q 19/10

(52) U.S. Cl. CPC A61K 8/66; A61K 8/41; A61K 8/60; A61Q 19/10

(57) ABSTRACT

Body wash compositions having an increased shelf-life are described. The body wash compositions include a Maillard reaction inhibiting enzyme that prevents a Maillard reaction between an amine and a reducing sugar. The Maillard reaction can cause yellowing of the body wash compositions. The body wash compositions which includes the Maillard reaction inhibiting enzyme do not undergo yellowing and thus have an increased shelf-life compared to equivalent body wash compositions that lack such an enzyme.

### CROSS-REFERENCE TO RELATED APPLICATION

This application claims the benefit of U.S. Provisional Patent Application 63/619,586, filed Jan. 10, 2024, the entire contents of which are incorporated by reference herein.

### BACKGROUND OF THE INVENTION

The present disclosure relates to a personal care composition, and more particularly to body wash compositions having an increased shelf-life.

In body wash compositions, there is an ongoing trend to include sustainable materials in formulating the body wash compositions. Sustainable materials are derived from renewable, recycled and waste carbon sources and their combination, which at the end of life can be recycled, biodegraded, or composted. Sustainable materials also exhibit reduced environmental impact throughout their life cycle. The use of sustainable materials within body wash compositions can reduce the cost of formulating the body wash and can provide an environmentally friendly product.

Reducing sugars undergo a Maillard reaction and cause browning in products when in the presence of an amine and heat. Amines can be found in body wash compositions from a variety of sources including, for example, proteins and surfactants such as quaternary ammonium compounds, cetrimonium chloride, dimethyl myristamine and lauramidopropylamine oxide. Browning reactions cause body wash compositions to turn yellow over time, especially when stored at elevated temperatures such as, for example, from 35° C. to 50° C. In some cases, it has been observed that as little as 40 ppm of reducing sugars can cause browning reactions to occur within a body wash composition over time.

The discoloration, i.e., yellowing, of body wash compositions caused by the Maillard reaction can lead to consumer dissatisfaction and consumer apprehension that the body wash composition is no longer useable. Thus, there is a need for providing non-yellowing body wash compositions.

### SUMMARY OF THE INVENTION

According to various aspects according to the disclosure, body wash compositions having increased shelf-lives. Body wash compositions according to various aspects of the disclosure include an enzyme that prevents or inhibits a Maillard reaction from occurring in said body wash compositions. This enzyme is referred to herein as a “Maillard reaction inhibiting enzyme”. A Maillard reaction is a chemical reaction between an amine and a reducing sugar, which can create melanoidins. Melanoidins are brown, high molecular weight heterogeneous polymers that are formed when sugars and amine source combine through the Maillard reaction. The formation of such polymers can cause browning to occur within a body wash composition. Body wash compositions according to the disclosure which include the Maillard reaction inhibiting enzyme do not undergo yellowing and thus exhibit an increased shelf-life compared to equivalent body wash compositions that lack such an enzyme. Due to the prevention that would regularly occur prior to the use of said composition by a consumer, consumer confidence in body wash compositions according to the disclosure will be greater than for equivalent body wash compositions that lack a Maillard reaction inhibiting enzyme.

In some instances, body wash compositions according to various aspects of the disclosure comprise, consist essentially of, or consist of, at least one surfactant; an amine source; sustainable raw material including a reducing sugar; a Maillard reaction inhibiting enzyme that inhibits reaction between the amine source and the reducing sugar; and at least 40 weight percent of water, based on the total weight of the body wash composition. In some instances, body wash compositions according to the disclosure have a pH ranging from 5 to 6.5.

In some instances, the at least one surfactant of body soap compositions according to the disclosure comprises a primary surfactant. In some instances, the primary surfactant is a key foaming/cleaning agent in the body wash composition. In some instances, the primary surfactant comprises, consists essentially of, or consists of at least one anionic surfactant. In some instances, the primary surfactant is present in an amount from about 1 weight percent to about 40 weight percent, based on the total weight of the body wash composition.

In some instances, the at least one surfactant of body soap compositions according to the disclosure comprises a primary surfactant and a secondary surfactant. In some instances, the secondary surfactant is a surfactant that is used to reduce the drying effect of the primary surfactant and modify the aesthetic properties of the body wash composition. In some instances, the secondary surfactant comprises, consists essentially of, or consists of an amphoteric surfactant, a zwitterionic surfactant, or any combination thereof. In some instances, the secondary surfactant is present in an amount from about 0.1 weight percent to about 15 weight percent, based on the total weight of the body wash composition.

In some instances, the amine source of body soap compositions according to the disclosure comprises a protein, an amine containing surfactant, a chelating agent, an amine containing enzyme, hydrolyzed protein or any combination thereof.

In some instances, the amine source of body wash compositions according to the disclosure is present in an amount from about 0.05 weight percent to about 30 weight percent, based on the total weight of the body wash composition.

In some instances, the sustainable raw of body wash compositions according to the disclosure material comprises a sustainable polymer, a sustainable solvent or a combination of a sustainable polymer and a sustainable solvent.

In some instances, the sustainable raw material of body wash compositions according to the disclosure is a sustainable solvent, where the sustainable solvent comprises, consists essentially of, or consists of, sorbitol, xylitol, mannitol, lactitol, isomalt, maltitol, a hydrogenated starch hydrolysate, sucrose, stachyose, verbascose, trehalose, raffinose or combinations thereof.

In some instances, the sustainable raw material of body wash compositions according to the disclosure is a sustainable polymer, where the sustainable polymer comprises a starch, a glycogen, a galactogen, a cellulose, a chitin or mixtures thereof.

In some instances, the Maillard rection inhibiting enzyme comprises hexose oxidase, fructosamine oxidase, fructosamine kinase, carbohydrate oxidase or combinations thereof.

In some instances, the Maillard reaction inhibiting enzyme is present in an amount of from about 0.001 weight percent to about 3 weight percent, based on the total weight of the composition.

In some instances, body wash compositions according to various aspects of the disclosure comprises, consists essentially of, or consists of, at least one surfactant; a primary amine source comprising a fermented raw material that includes a reducing sugar; a Maillard reaction inhibiting enzyme that inhibits reaction between the primary amine source and the reducing sugar; and at least 40 weight percent water, based on the total weight of the composition, wherein the composition has a pH from 5 to 6.5.

In some instances, where body wash compositions according to the disclosure include a primary amine source, the at least one surfactant may comprise a primary surfactant. In some instances, the primary surfactant is present in an amount from about 1 weight percent to about 40 weight percent, based on the total weight of the body wash composition.

In some instances, where body wash compositions according to the disclosure include a primary amine source, the at least one surfactant may comprise a primary surfactant and a secondary surfactant. In some instances, the secondary surfactant is present in an amount from about 0.1 weight percent to about 15 weight percent, based on the total weight of the body wash composition.

In some instances, where body wash compositions according to the disclosure include a primary amine source, the primary amine source comprises a biosurfactant such as, for example, sophorolipid or rhamnolipid.

In some instances, where body wash compositions according to the disclosure include a primary amine source, the primary amine source is present in an amount from about 0.05 weight percent to about 30 weight percent, based on the total weight of the body wash composition.

In some instances, where body wash compositions comprise an amine source, the Maillard rection inhibiting enzyme comprises, consists essentially of, or consists of fructosamine oxidase, fructosamine kinase, carbohydrate oxidase or combinations thereof.

In some instances, where body wash compositions comprise an amine source, the Maillard inhibiting enzyme is present in an amount of from about 0.001 weight percent to about 3 weight percent, based on the total weight of the body wash composition.

In some instances, where body wash compositions comprise an amine source, the body wash composition can further comprise, consist essentially of, or consist a sustainable raw material including a reducing sugar. In some instances, the sustainable raw material including the reducing sugar can include a sustainable polymer, a sustainable solvent or a combination of a sustainable polymer and a sustainable solvent. In some instances, the sustainable polymer comprises a starch, a glycogen, a galactogen, a cellulose, a chitin or mixtures thereof. In some embodiments, the sustainable solvent comprises sorbitol, xylitol, mannitol, lactitol, isomalt, maltitol, a hydrogenated starch hydrolysate, sucrose, stachyose, verbascose, trehalose, raffinose or combinations thereof.

In some instances, the sustainable material (i.e., raw material) used in formulating the body wash composition comprises, consists essentially of, or consists of a reducing sugar, such as, for example, fructose or glucose. In some instances, the reducing sugar can be a contaminant (i.e., residual) in the sustainable material or it can be part (i.e., a moiety) of the sustainable material, for example, a monomer of a polymer, which degrades from the sustainable material over time.

Various non-limiting inventive aspects according to the disclosure are as follows.

In some instances, a first aspect of the disclosure can be described as body wash composition comprising at least one surfactant, an amine source, a sustainable raw material including a reducing sugar, a Maillard reaction inhibiting enzyme that inhibits reaction between the amine source and the reducing sugar, and at least 40 percent water, based on the total weight of the body wash compositions.

In some instances, a second aspect of the disclosure can be described as a body wash composition according to the first aspect, wherein the body wash composition has a pH ranging from about 5 to about 6.5.

In some instances, a third aspect of the disclosure can be described as a body wash composition according to the first aspect or second aspect, wherein the at least one surfactant comprises a primary surfactant.

In some instances, a fourth aspect of the disclosure can be described as a body wash composition according to the third aspect, wherein the primary surfactant is present in an amount ranging from about 1 weight percent to about 40 weight percent, based on the total weight of the body wash compositions.

In some instances, a fifth aspect of the disclosure can be described as a body wash composition according to the third or fourth aspect, wherein the at least one surfactant further comprises a secondary surfactant.

In some instances, a sixth aspect of the disclosure can be described as a body wash composition according to the fifth aspect, wherein the secondary surfactant is present in an amount ranging from about 0.1 weight percent to about 15 weight percent, based on the total weight of the body composition.

In some instances, a seventh aspect of the disclosure can be described as a body wash composition according to any one of the first through the sixth aspects, wherein the amine source comprises a protein, an amine containing surfactant, a chelating agent, an amine containing enzyme, or a hydrolyzed protein, or any combination thereof.

In some instances, an eighth aspect of the disclosure can be described as a body wash composition according to any one of the first through the seventh aspects, wherein the amine source is present in an amount ranging from about 0.05 weight percent to about 30 weight percent, based on the total weight of the body wash composition.

In some instances, a ninth aspect of the disclosure can be described as a body wash composition according to any one of the first through the eighth aspects, wherein the sustainable raw material comprises a sustainable polymer or a sustainable solvent, or a combination of a sustainable polymer and a sustainable solvent.

In some instances, a tenth aspect of the disclosure can be described as a body wash composition according to the ninth aspect, wherein the sustainable solvent comprises sorbitol, xylitol, mannitol, lactitol, isomalt, maltitol, a hydrogenated starch hydrolysate, sucrose, stachyose, verbascose, trehalose, or raffinose, or any combination thereof.

In some instances, an eleventh aspect of the disclosure can be described as a body wash composition according to the ninth aspect, wherein the sustainable polymer comprises a starch, a glycogen, a galactogen, a cellulose, or a chitin, or any combination thereof.

In some instances, a twelfth aspect of the disclosure can be described as a body wash composition according to any one of the first through the eleventh aspects, wherein the Maillard rection inhibiting enzyme comprises fructosamine oxidase, fructosamine kinase, or carbohydrate oxidase, or any combination thereof.

In some instances, a thirteenth aspect of the disclosure can be described as a body wash composition according to any one of the first through the twelfth aspects, wherein the Maillard reaction inhibiting enzyme is present in an amount of from about 0.001 weight percent to about 3 weight percent, based on the total weight of the composition.

In some instances, a fourteenth aspect of the disclosure can be described as a body wash composition comprising, at least one surfactant, a primary amine source comprising a fermented raw material that includes a reducing sugar, a Maillard reaction inhibiting enzyme that inhibits reaction between the primary amine source and the reducing sugar, and at least 40 weight percent water, based on the total weight of the composition.

In some instances, a fifteenth aspect of the disclosure can be described as a body wash composition according to the fourteenth aspect, wherein the body wash composition has a pH ranging from about 5 to about 6.5.

In some instances, a sixteenth aspect of the disclosure can be described as a body wash composition according to the fourteenth of fifteenth aspect, wherein the at least one surfactant comprises a primary surfactant.

In some instances, a seventeenth aspect of the disclosure can be described as a body wash composition according to the sixteenth aspect, wherein the primary surfactant is present in an amount ranging from about 1 weight percent to about 40 weight percent, based on the total weight of the body wash composition.

In some instances, an eighteenth aspect of the disclosure can be described as a body wash composition according to either of the sixteenth and seventeenth aspects, wherein the at least one surfactant further comprises a secondary surfactant.

In some instances, a nineteenth aspect of the disclosure can be described as a body wash composition according to the eighteenth aspect, wherein the secondary surfactant is present in an amount ranging from about 0.1 weight percent to about 15 weight percent, based on the total weight of the body wash composition.

In some instances, a twentieth aspect of the disclosure can be described as a body wash composition according to any one of the fourteenth to the nineteenth aspects, wherein the primary amine source comprises a biosurfactant.

In some instances, a twenty-first aspect of the disclosure can be described as a body wash composition according to any one of the fourteenth to the twentieth aspects, wherein the primary amine source is present in an amount from about 0.05 weight percent to about 30 weight percent, based on the total weight of the body wash composition.

In some instances, a twenty-second aspect of the disclosure can be described as a body wash composition according to any one of the fourteenth to the twenty-first aspects, wherein the Maillard rection inhibiting enzyme comprises fructosamine oxidase, fructosamine kinase, or carbohydrate oxidase, or any combination thereof.

In some instances, a twenty-third aspect of the disclosure can be described as a body wash composition according to any one of the fourteenth to the twenty-second aspects, wherein the Maillard inhibiting enzyme is present in the composition in an amount of from about 0.001 weight percent to about 3 weight percent, based on the total weight of the body wash composition.

In some instances, a twenty-fourth aspect of the disclosure can be described as a body wash composition according to any one of the fourteenth to the twenty-third aspects, wherein the body wash composition further comprises a sustainable solvent including a reducing sugar or a sustainable polymer including a reducing sugar, or a combination of a sustainable polymer and a sustainable solvent.

In some instances, a twenty-fifth aspect of the disclosure can be described as a body wash composition according to the twenty-fourth aspect, wherein the sustainable solvent comprises sorbitol, xylitol, mannitol, lactitol, isomalt, maltitol, a hydrogenated starch hydrolysate, sucrose, stachyose, verbascose, trehalose, or raffinose, or any combination thereof.

In some instances, a twenty-sixth aspect of the disclosure can be described as a body wash composition according to the twenty-fourth aspect, wherein the sustainable polymer comprises a starch, a glycogen, a galactogen, a cellulose, or a chitin, or any combination thereof.

### BRIEF DESCRIPTION OF THE DRAWINGS

FIG. 1 is an image depicting an exemplary pantone scale for characterization as to the degree of yellowing of detergent compositions comprising reducing sugar agents.

### DETAILED DESCRIPTION

The present disclosure will now be described in greater detail by referring to the following discussion and drawings that accompany the present disclosure. In the following description, numerous specific details are set forth, such as particular structures, components, materials, dimensions, processing steps and techniques, in order to provide an understanding of the various embodiments according to the disclosure. However, it will be appreciated by one of ordinary skill in the art that the various embodiments according to the disclosure may be practiced without these specific details. As used throughout the present disclosure, the term “about” generally indicates no more than ±10%, ±5%, ±2%, ±1% or ±0.5% from a number. When a range is expressed in the present disclosure as being from one number to another number (e.g., 20 to 40), the present disclose contemplates any numerical value that is within the range (i.e., 22, 24, 26, 28.5, 31, 33.5, 35, 37.7, 39 or 40) or any in amount that is bounded by any of the two values that can be present within the range (e.g., 28.5-35).

The terminology used herein is for the purpose of describing particular embodiments only and is not intended to be limiting according to the disclosure. As used herein, the singular forms “a”, “an” and “the” are intended to include the plural forms as well, unless the context clearly indicates otherwise. It will be further understood that the terms “comprises” and/or “comprising”, when used in this disclosure, specify the presence of stated features, integers, steps, operations, elements, and/or components, but do not preclude the presence or addition of one or more other features, integers, steps, operations, elements, components, and/or groups thereof.

Various aspects of the disclosure are directed towards body wash compositions that have an increased shelf-life. Body wash compositions according to various aspects of the disclosure include a Maillard reaction inhibiting enzyme that inhibits a reaction between an amine and a reducing sugar. The presence of such an enzyme inhibits the Maillard reaction between the reducing sugar and the amine and thus the body wash compositions according to the disclosure have an increased shelf-life. The increased shelf-life is evidenced by the absence of yellowing of the body wash compositions over an extended period of time. In some instances, the enzyme inhibits the reaction of the reducing sugar with the amine throughout the entire shelf-life of the body wash composition including at the beginning of the formulation process. These and other aspects according to the disclosure will now be described in greater detail.

Body wash compositions according to various aspects of the disclosure comprise at least one surfactant. In some instances, the surfactant is an anionic surfactant, a nonionic surfactant, a cationic surfactant, an amphoteric surfactant, a zwitterionic surfactant or any combination such surfactants. In some instances, the at least one surfactant can include a primary surfactant and optionally a secondary surfactant. In some instances, the primary surfactant is a key foaming/cleaning agent in the body wash composition. In some instances, the primary surfactant comprises at least one anionic surfactant. In some instances, the secondary surfactant is a surfactant that is used to reduce the drying effect of the primary surfactant and modify the aesthetic properties of the body wash composition. In some instances, the secondary surfactant comprises an amphoteric surfactant, a zwitterionic surfactant, or any combination thereof.

In some instances, the surfactant (primary or secondary) that can be included in the body wash composition includes any surfactant that is dermatologically compatible with skin and/or hair. By “dermatologically compatible” it is meant that the compound does not cause irritation when applied to the skin and/or hair for the purpose of cleaning and then is subsequently removed within about 30 minutes of application.

In some instances, the primary surfactant is preferably present at a level of from about 1 weight percent to about 40 weight percent, more preferably from about 3 weight percent to about 30 weight percent, and most preferably from about 5 weight percent to about 20 weight percent, based on the total weight of the body wash composition. In some instances, the secondary surfactant is preferably present at a level of from about 0.1 weight percent to about 15 weight percent, more preferably from about 0.2 weight percent to about 10 weight percent, and most preferably from about 0.5 weight percent to about 5 weight percent based on the total weight of the body wash composition.

According to various aspects of the disclosure, anionic surfactants useful in body wash compositions according to the disclosure include, for example, alkyl sulfates, alkyl ether sulfates, alkyl ether carboxylates, acyl isethionates, acyl sarcosinates, acyl taurines, or alkali metal, ammonium, or alkanolammonium salts thereof, or combinations thereof. In some instances, the alkyl or acyl groups of such anionic surfactants contain from about 10 to about 20 carbon atoms per alkyl or acyl group, and in some instances, preferably from about 12 to about 18 carbon atoms per alkyl or acyl group. In some instances, the alkyl or acyl groups may be linear, branched, saturated or unsaturated or combinations thereof. In some instances, the use of linear alkyl or acyl groups is preferred. A further description of anionic surfactants that can be use in the body wash composition is provided herein below.

According to various aspects of the disclosure, the anionic surfactant includes at least one alkyl sulfate, alkyl ether sulfate, salts thereof, or combinations thereof. In some instances, these surfactants have respectively the general formula (I) and/or the general formula (II):

R₉OSO₃⁻M⁺ (I)

R₉O(C₂H₄O)ₓSO₃⁻M⁺ (II)

where R₉ is an alkyl group of about 10 to about 20 carbon atoms, x is an integer ranging from 1 to about 10, and M⁺ is a water-soluble cation such as an ammonium ion, an alkylammonium ion (such as triethylammonium or triethanolammonium ion), or an alkali metal ion such as sodium or potassium. In some instances, R₉ is preferably an alkyl group of about 12 to about 18 carbon atoms. In some instances, x is preferably an integer ranging from 1 to about 4. In some instances, R₉ is preferably an alkyl group of about 12 to about 18 carbon atoms and x is preferably an integer ranging from 1 to about 4.

Non-limiting examples of preferred anionic surfactants of general formula I and/or II include ammonium lauryl sulfate, sodium lauryl sulfate, ammonium laureth sulfate (which may preferably have 1 to about 3 moles ethylene oxide), sodium laureth sulfate (which may preferably have 1 to about 3 moles ethylene oxide), or combinations thereof.

Other suitable anionic surfactants that can be employed in body wash compositions according to the disclosure are the water-soluble salts of organic, sulfuric acid reaction products conforming to the formula [R¹—SO₃-M] where R¹ is a straight or branched chain, saturated, aliphatic hydrocarbon radical having from about 8 to about 24, preferably from about 10 to about 18, carbon atoms; and M is a cation described hereinbefore. Non-limiting examples of such surfactants are the salts of an organic sulfuric acid reaction product of a hydrocarbon of the methane series, including iso-, neo-, and n-paraffins, having from about 8 to about 24 carbon atoms, preferably about 12 to about 18 carbon atoms and a sulfonating agent, e.g., SO₃, H₂SO₄, obtained according to known sulfonation methods, including bleaching and hydrolysis. In some instances, preferred are alkali metal and ammonium sulfonated C₁₀ to C₁₈ n-paraffins.

In some instances, other suitable anionic surfactants that can be employed in body wash compositions according to the disclosure are the reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil. Other similar anionic surfactants are described in U.S. Pat. Nos. 2,486,921; 2,486,922; and 2,396,278, the entire contents of each of which are incorporated herein by reference.

In some instances, suitable anionic surfactants include succinates, examples of which include disodium N-octadecylsulfosuccinate; disodium lauryl sulfosuccinate; diammonium lauryl sulfosuccinate; tetrasodium N-(1,2-dicarboxyethyl)-N-octadecylsulfosuccinate; diamyl ester of sodium sulfosuccinic acid; dihexyl ester of sodium sulfosuccinic acid; and dioctyl esters of sodium sulfosuccinic acid.

Additional suitable anionic surfactants that can be employed in body wash compositions according to the disclosure include olefin sulfonates having about 10 to about 24 carbon atoms. In this context, the term “olefin sulfonates” refers to compounds which can be produced by the sulfonation of alpha-olefins by means of uncomplexed sulfur trioxide, followed by neutralization of the acid reaction mixture in conditions such that any sulfonates which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form. The alpha-olefins from which the olefin sulfonates are derived are mono-olefins having from about 10 to about 24 carbon atoms, preferably from about 12 to about 16 carbon atoms. In some instances, they are preferably straight chain olefins. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process. A non-limiting example of such an alpha-olefin sulfonate mixture is described in U.S. Pat. No. 3,332,880, the entire contents of which are incorporated herein by reference.

In some instances, a class of anionic surfactants that can be employed in body wash compositions according to the disclosure are the beta-alkyloxy alkane sulfonates. These surfactants conform to formula (III):

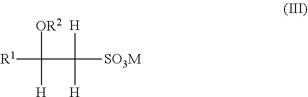

where R¹ is a straight chain alkyl group having from about 6 to about 20 carbon atoms, R² is a lower alkyl group having from about 1 to about 3 carbon atoms, preferably 1 carbon atom, and M is a water-soluble cation as described hereinbefore. In some instances, preferred anionic surfactants for use in body wash compositions according to the disclosure include sodium tridecyl benzene sulfonate and sodium dodecyl benzene sulfonate.

In some instances, the sulfates according to formula I, the sulfates according to formula II, the sulfonates of formula (III) and any combination of these anionic surfactants are preferred anionic surfactants that can be used in body wash compositions according to the disclosure as the primary surfactants.

In some instances, body wash compositions according to the disclosure comprise (in addition to an anionic surfactant) a secondary surfactant that includes an amphoteric surfactant, zwitterionic surfactant, or any combinations thereof. In some instances, preferably, the total amount of amphoteric and zwitterionic surfactant is from 0 to about 5 weight percent and more preferably from about 1 to about 4 weight percent, based on the total weight of the body wash composition.

Non-limiting examples of suitable amphoteric or zwitterionic surfactants include, but are not limited to, alkyl betaines, alkylamidobetaines, aminopropionates, aminoglycinates, imidazolinium betaines, sulfobetaines, or any combination thereof.

In some instances, preferred zwitterionic surfactants include compounds according to formula (IV):

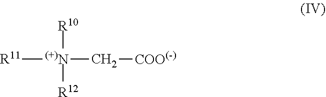

where R¹¹ is an alkyl or alkenyl group containing from about 10 to about 20 carbon atoms or a R¹³—CONH—(CH₂)ₚ group, where R¹³ is an alkyl or alkenyl group containing from about 10 to about 20 carbon atoms and p is an integer ranging from about 2 to about 5, and R¹⁰ and R¹² are alkyl groups containing from 1 to about 5 carbon atoms or hydroxyalkyl groups containing from about 2 to about 4 carbon atoms.

In some instances, preferred amphoteric surfactants include compounds according to formula (V):

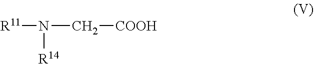

where R¹¹ is an alkyl or alkenyl group containing from about 10 to about 20 carbon atoms or a R¹³—CONH—(CH₂)ₚ group, where R¹³ is an alkyl or alkenyl group containing from about 10 to about 20 carbon atoms and p is an integer ranging of from about 2 to about 5, and R¹⁴ is an alkyl group containing from 1 to about 5 carbon atoms or a hydroxyalkyl group containing from about 2 to about 4 carbon atoms.

Non-limiting examples of amphoteric or zwitterionic surfactants include sodium 3-dodecyl-aminopropionate, sodium 3-dodecylaminopropane sulfonate, sodium lauroamphoacetate, coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alpha-carboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, oleamidopropyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine or combinations thereof. Most preferably, the zwitterionic or amphoteric surfactant is cocoamidopropyl betaine, oleamidopropyl betaine, or a combination thereof.

In some instances, other common cosmetic additives may be included in body wash compositions according to the disclosure, as long as the basic properties of the body wash composition are not adversely affected. Such optional cosmetic additives may be found, for example, in the International Cosmetic Ingredients Dictionary, 7th Edition, 1997, published by the CTFA in Washington D.C, the entire contents of which are incorporated by reference herein. Preferably, these optional components, in total, are present in an amount of from 0 to 20 weight percent, and more preferably from about 0.1 to about 12 weight percent, based on the total weight of the composition.

Non-limiting examples of optional additive components include, but are not limited to, one or more surfactants such as nonionic surfactants or cationic surfactants, protein hydrolyzates, other conditioning agents, inorganic electrolyte salts, or any combination thereof.

In some instances, one type of nonionic surfactant that can be employed in body wash compositions according to the disclosure is amides. In some instances, amides, including alkanol amides, are the condensation products of fatty acids with primary and secondary amines or alkanolamines to yield products of the general formula (VI):

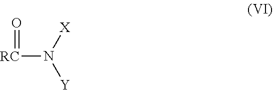

wherein R—C(═O)— is a fatty acid radical and R is C₈₋₂₀; X is an alkyl, aromatic or alkanol (CHR′CH₂OH wherein R′ is H or C₁₋₆ alkyl); Y is H, alkyl, alkanol or X. Suitable amides include, but are not limited to, cocamide, lauramide, oleamide and stearamide. Suitable alkanolamides include, but are not limited to, cocamide DEA, cocamide MEA, cocamide MIPA, isostearamide DEA, isostearamide MEA, isostearamide MIPA, lanolinamide DEA, lauramide DEA, lauramide MEA, lauramide MIPA, linoleamide DEA, linoleamide MEA, linoleamide MIPA, myristamide DEA, myristamide MEA, myristamide MIPA, Oleamide DEA, Oleamide MEA, Oleamide MIPA, palmamide DEA, palmamide MEA, palmamide MIPA, palmitamide DEA, palmitamide MEA, palm kernelamide DEA, palm kernelamide MEA, palm kernelamide MIPA, peanutamide MEA, peanutamide MIPA, soyamide DEA, stearamide DEA, stearamide MEA, stearamide MIPA, tallamide DEA, tallowamide DEA, tallowamide MEA, undecylenamide DEA, undecylenamide MEA and PPG-2 hydrodroxyethyl coco/isostearamide. The condensation reaction may be carried out with free fatty acids or with all types of esters of the fatty acids, such as fats and oils, and particularly methyl esters. The reaction conditions and the raw material sources determine the blend of materials in the end product and the nature of any impurities.

In some instances, other types of surfactants, not previously described herein, that are typically used in compositions may optionally be added to body wash compositions according to the disclosure such as nonionic and/or cationic surfactants. In some instances, these surfactants are preferably present in an amount of no more than about 10 weight percent, more preferably in an amount of 0 weight percent to about 5 weight percent, and most preferably in an amount of about 0.01 weight percent to about 3 weight percent, based on the total weight of the composition. In some instances, the body wash composition comprises nonionic surfactants present in an amount preferably ranging from 0 weight percent to about 10 weight percent and more preferably from 0 weight percent to about 2 weight percent, based on the total weight of the body wash composition. Non-limiting examples of nonionic surfactants include alkyl polyglycosides, alkyl oligoglycosides, ethoxylated fatty alcohols, sorbitan esters, or alkanolamides such as cocamide MEA, cocamide DEA, soyamide DEA, lauramide DEA, lauramide MEA, lauramide MIPA, oleamide MIPA, ricinoleamide DEA, stearamide MEA, stearamide DEA, isostearamide MEA, isostearamide DEA, myristamide MEA, lauramide MEA, capramide DEA, stearamide DEA, oleylamide DEA, myristamide DEA, or tallowamide DEA, or combinations thereof.

In some instances, body wash compositions according to the disclosure comprise cationic surfactants present in an amount preferably ranging from 0 weight percent to about 5 weight percent and more preferably from 0 weight percent to about 1 weight percent, based on the total weight of the composition. Examples of cationic surfactants include quaternary ammonium compounds.

A more extensive list of surfactants is described for example in U.S. Pat. No. 5,955,066, and McCutcheon's Emulsifiers & Detergents, North American Edition and International Edition, 1999 Annuals, published by McCutcheon's Division, MC Publishing Company, Glen Rock, N.J. (1999). The complete disclosure of these documents are incorporated herein by reference in their entireties.

Body wash compositions according to various aspects of the disclosure are preferably formulated with water as a carrier. In some instances, water is present in an amount of at least about 40 weight percent, preferably from about 50 weight percent to about 90 weight percent, and more preferably from about 60 weight percent to about 80 weight percent, based on the total weight of the body wash composition.

In some instances, body wash compositions according to the disclosure may further comprise other carriers such as organic solvents. In some instances, the organic solvents are present in body wash compositions in an amount of no more than about 5 weight percent and more preferably in an amount of from about 0.1 to about 1 weight percent based on the total weight of the body wash composition. Non-limiting examples of organic solvents include C₂ to C₆ monoalcohols, such as ethanol, propanol, isopropanol, or tert-butyl alcohol; ethylene glycol; ethylene glycol monomethyl ether; ethylene glycol monoethyl ether; ethylene glycol monobutyl ether; ethylene glycol monoethyl ether acetate; polyethylene glycol, propylene glycol; propylene glycol monomethyl ether; dipropylene glycol monomethyl ether; glycerol; diethylene glycol; glycerin or combinations thereof. In some instances, preferably, body wash compositions according to the disclosure are formulated as monoalcohol-free compositions.

In some instances, body wash compositions according to the disclosure further comprise an amine source and a sustainable raw material including a reducing sugar. In some instances, the amine source includes any compound that contains at least one amine functional group and that can be used in body wash compositions. For example, the amine source can include a protein, an amine containing surfactant (e.g., for example, a quaternary ammonium compound, an ethanolamine compound [e.g., monoethanolamine, diethanolamine, triethanolamine or mixtures thereof], cetrimonium chloride, dimethyl myristamine and lauramidopropylamine oxide), a chelating agent (such as, for example, ethylenediamine, ethylenediaminetetraacetic (EDTA)), an amine containing enzyme, a hydrolyzed protein (such as, for example, hydrolyzed keratin or hydrolyzed wheat proteins), or any combination thereof. In some instances, the amine source is preferably present in body wash compositions in an amount from about 0.05 weight percent to about 30 weight percent, with an amount from about 0.25 weight percent to about 20 weight percent being more preferable, and an amount from about 0.5 weight percent to about 15 weight percent being even more preferable based on the total weight of the body wash composition. In some instances in which the amine source is an amine containing surfactant, the amine containing surfactant is preferably present in body wash compositions in an amount from about 1 weight percent to about 20 weight percent. In some instances in which the amine source is other than an amine containing surfactant, the non-surfactant amine source is preferably present in body wash composition in an amount from about 0.05 weight percent to about 5 weight percent based on the total weight of the body wash composition.

Ethanolamine compounds contain a group of amino alcohols. Ethanolamine compounds can be used as a surfactant in body wash compositions according to the disclosure. Ethanolamine compounds can aid in the removal of dirt, grease and stains. Illustrative examples of ethanolamine compounds include, but are not limited to, monoethanolamine, diethanolamine, triethanolamine or mixtures thereof. In some instances, the ethanolamine is present in an amount ranging from about 0.5 to about 10 wt % of the body wash composition. In some instances, the ethanolamine is present in an amount ranging from about 1.0 to about 5.0 wt % of the body wash composition.

A chelating agent is a compound containing a ligand (typically organic) that can react with metal ions to form a stable, water-soluble complex. Chelating agents can also be referred to as chelants, chelators or sequestering agents. Chelating agents can have a ring-like center which forms at least two bonds with the metal ion. In some instances, the chelate is any compound that includes at least an amine functional and that can be used in body wash formulations. In some instances, the chelating agent is present in an amount ranging from about 0.1 to about 5 wt % of the body wash composition. In some instances, the chelating agent is present in an amount ranging from about 0.1 to about 2.5 wt % of the body wash composition. Exemplary chelates that can be employed as an amine source include, but are not limited to, tetrasodium iminodisuccinate, ethylenediamine, ethylenediaminetetraacetic (EDTA) or mixtures thereof. In some instances, the chelating agent is N,N-bis(carboxymethyl)-L-glutamic acid tetrasodium salt. Chelating agents are sometimes used as water softeners in body wash compositions. In some instances, the chelating agent is iminodisuccinate (IDS), ethylenediaminetetraacetic acid (EDTA), nitrilotriacetic acid, diethylenetriaminepenta(methylenephosphonic acid), nitrilotris(methylenephosphonic acid), ethylenediamine-N,N′-disuccinic acid (EDDS), hydroxyethylenediaminetriacetic acid (HEDTA), N,N-bis(carboxymethyl)-L-glutamic acid tetrasodium salt, Alanine, N,N-bis(carboxymethyl)-alanine, or other chelating compounds comprising an amine group.

Amino acids are organic compounds that contain both amino and carboxylic functional groups. Exemplary amino acids that can be used as the amine source include, but are not limited to, arginine, histidine, lysine, aspartic acid, glutamic acid, serine, threonine, asparagine, glutamine, cysteine, selenocysteine, glycine, proline, alanine, valine, isoleucine, methionine, phenylalanine, tyrosine, tryptophan or any combination thereof.

In some instances, amine-containing enzymes (i.e., enzymes that containing at least one amine functionality) can also be used as the amine source.

In some instances, body wash compositions according to the disclosure comprise a sustainable raw material including a reducing sugar. In some instances, these reducing sugars can be a residual contaminant present in the sustainable raw material, or it can be a moiety of sustainable raw material that can be released upon degradation. For example, a reducing sugar can be contained in a sustainable solvent and/or sustainable polymer that can be used in formulating a body wash composition according to the disclosure. In some instances, body wash compositions according to the disclosure further comprise a sustainable polymer that includes a reducing sugar monomer. In some instances, the sustainable polymer can be present in body wash compositions in an amount of from about 0.01 weight percent to about 10 weight percent based on the total weight percentage of the body wash composition. In some instances, the sustainable polymer can be present in the body wash composition in an amount of from about 0.25 weight percent to about 2 weight percent based on the total weight percentage of the body wash composition. In some instances, the sustainable polymer can be present in the body wash composition in an amount of from about 0.5 weight percent to about 1 weight percent based on the total weight percentage of the body wash composition. In some instances, the sustainable polymer including the reducing sugar can include a polysaccharide.

A polysaccharide is a polymer made of chains of monosaccharides that are joined by glycosidic linkages. A polysaccharide typically consists of more than ten monosaccharide units. Polysaccharides may be linear or branched. Linear polysaccharides are typically rigid polymers, while branched polysaccharides are typically soluble in water. In some instances, a polysaccharide comprises starches, glycogens, galactogens, cellulose and/or chitin. An illustrative example of a polysaccharide that can be used in body compositions according to the disclosure as the sustainable polymer includes, but is not limited to, poly alpha-1,6-glucan.

In some instances, the sustainable polymer comprises a reducing sugar monomer. In some instances the reducing sugar monomer can be a contaminate (residual) or it can be a moiety of the sustainable polymer that can be released upon degradation. In some instances, in body wash compositions including an amine source, the reducing sugar monomer (residual or released) and the amine source can react together under heat to cause unwanted yellowing of the body wash composition. In some instances, the reducing sugar monomer is present in the sustainable polymer in an amount of from about 0.0001 weight percent to about 1 weight percent, with an amount of from about 0.005 weight percent to about 0.5 weight percent being more preferable. A reducing sugar monomer is any sugar monomer that can act as a reducing agent. The reducing sugar monomer can include a monosaccharide or disaccharide. Exemplary reducing sugar monomers that can be contained in the polymer include, but are not limited to, fructose, glucose, hexose, lactose, glyceraldehyde, arabinose, maltose, galactose, ribose, xylose, cellobiose or combinations thereof. The reducing sugar monomer can be present in the raw materials that are used in forming the sustainable polymer mentioned above.

In some instances, body wash compositions according to the disclosure comprise at least one sustainable solvent that includes a reducing sugar (residual or moiety). In some instances, the sustainable solvent is preferably present in body wash compositions in an amount of from about 0.01 weight percent to about 60 weight percent, with an amount of from about 1 weight percent to about 30 weight percent being more preferable, and an amount of from about 5 weight percent to about 20 weight percent being even more preferable based on the total weight of the body wash composition. Illustrative sustainable solvents including a reducing sugar that can be employed in the present disclosure include, but are not limited to, sorbitol, xylitol, mannitol, lactitol, isomalt, maltitol, a hydrogenated starch hydrolysate, sucrose, stachyose, verbascose, trehalose, raffinose or combinations thereof. In some instances, the reducing sugar is present in the sustainable solvent in an amount of from about 0.0001 weight percent to about 1 weight percent, with an amount of from about 0.005 weight percent to about 0.5 weight percent being more preferable. The reducing sugar can be present in the raw materials that are used in forming the at least one sustainable solvent mentioned above.

In some instances, body wash compositions according to the disclosure comprise both a sustainable solvent that includes a reducing sugar (residual or moiety) and a sustainable polymer that includes a reducing sugar monomer (residual or moiety).

In some instances, body wash compositions according to the disclosure comprise a primary amine source comprising a fermented raw material that includes a reducing sugar. Examples of such primary amine sources include, but are not limited to, a biosurfactant such as, for example, sophorolipid or rhamnolipid. In some instances, the reducing sugar can be a contaminate (residual) or it can be a moiety of the primary amine source that can be released upon degradation. In body wash compositions including such a primary amine source, the reducing sugar (residual or released) and the amine present in the primary amine source can react together under heat to cause unwanted yellowing of the body wash composition.

In some instances, the reducing sugar is present in the primary amine source in an amount of from about 0.0001 weight percent to about 1 weight percent, with an amount of from about 0.005 weight percent to about 0.5 weight percent being more preferable. A reducing sugar is any sugar that can act as a reducing agent. The reducing sugar can include a monosaccharide or disaccharide. Exemplary reducing sugars that can be contained in the amine containing surfactant include, but are not limited to, fructose, glucose, hexose, lactose, glyceraldehyde, arabinose, maltose, galactose, ribose, xylose, cellobiose or combinations thereof. In some instances, the primary amine source is preferably present in the body wash composition in an amount from about 5 weight percent to about 30 weight percent, with an amount from about 10 weight percent to about 25 weight percent being more preferable, and an amount from about 12 weight percent to about 20 weight percent being even more preferable based on the total weight of the body wash composition.

In some instances, body wash compositions according to the disclosure comprise a primary amine source, a secondary amine source and a sustainable raw material including the reducing sugar as mentioned above. In some instances, the secondary amine source is an amine source in which non-fermented raw materials that do includes a reducing sugar are used in forming the same. In some instances, the secondary amine source comprises any compound that contains at least one amine functional group and that can be used in body wash compositions. For example, the amine source can include, but is not limited to, a protein, an amine containing surfactant (e.g., for example, a quaternary ammonium compound, an ethanolamine compound [e.g., monoethanolamine, diethanolamine, triethanolamine or mixtures thereof], cetrimonium chloride, dimethyl myristamine and lauramidopropylamine oxide), a chelating agent (such as, for example ethylenediamine, ethylenediaminetetraacetic (EDTA)), an amine containing enzyme, a hydrolyzed protein (such as, for example, hydrolyzed keratin or hydrolyzed wheat proteins) or any combination thereof. In some instances, the secondary amine source is preferably present in the body wash composition in an amount from about 0.05 weight percent to about 30 weight percent, with an amount from about 0.25 weight percent to about 20 weight percent being more preferable, and an amount from about 0.5 weight percent to about 15 weight percent being even more preferably based on the total weight percentage of the body wash composition.

Body wash compositions according to the disclosure comprise a Maillard reaction inhibiting enzyme. In some instances, the Maillard reaction inhibiting enzyme is any enzyme that can prevent yellowing of the body wash composition by inhibiting the reaction (i.e., Maillard reaction) between the reducing sugar and the amine source and/or between the amine and the reducing sugar of the amine containing surfactant. In some instances, the Maillard reaction inhibiting enzyme is preferably present in the body wash composition in an amount of from about 0.001 weight percent to about 3 weight percent, with an amount of from about 0.005 weight percent to about 0.5 weight percent being more preferably, and an amount from about 0.01 weight percent to about 0.1 weight percent being even more preferable based on the total weight of the body wash composition. In some instances, the Maillard reaction inhibiting enzyme is a reducing sugar oxidation enzyme. Such oxidation enzymes oxidize the reducing sugar that is present and prevent subsequent yellowing of the body wash composition over time. In some instances, the Maillard reaction inhibiting enzyme is an enzyme that reacts with fructose and glucose. Illustrative examples of Maillard reaction inhibiting enzymes that can prevent yellowing of the body wash composition according to the disclosure include, but are not limited to, fructosamine oxidase, fructosamine kinase, hexose oxidase, or carbohydrate oxidase. It is noted that within the range mentioned above, the Maillard reaction inhibiting enzyme can prevent the Maillard reaction from occurring throughout the entire shelf-life of the body wash composition.

The Maillard reaction inhibiting enzyme can be added to a raw material composition that contains at least one of the sustainable raw materials mentioned above utilizing techniques that are well known to those skilled in the art. In some instances, the addition includes metered addition. The addition can be performed with continuous stirring. In some instances, the addition is typically performed at or around room temperature, e.g., from about 20° C. to about 30° C. This addition provides the formulation in accordance with the present application. In addition to the Maillard reaction inhibiting enzyme and the raw material composition mentioned above, formulations according to the disclosure can include additional components as described below. The additional components can be present in a raw material composition that includes a sustainable raw material and a reducing sugar, or they can be added after forming the raw material composition.

Body wash composition according to the disclosure are formulated to have a pH that is dermatologically compatible with skin. In some instances, body wash compositions according to the disclosure are formulated to have a pH ranging from about 5 to about 6.5. In some instances, body wash compositions formulated to have a pH ranging from about 5.5 to about 6.0. In some instances, the pH of the body wash composition according to the disclosure can be adjusted with any of a number of commonly used cosmetic ingredients. Non-limiting examples of suitable pH adjusters include acids for decreasing the pH including inorganic acids such as phosphoric acid, nitric acid, or hydrochloric acid, or organic acids such as citric acid, lactic acid or tartaric acid; or basic compounds for increasing the pH, including organic amines such as triethanolamine, ethanolamine, or aminomethylpropanol, alkali metal or alkaline earth metal hydroxides such as sodium hydroxide or potassium hydroxide; ammonium hydroxide, basic amino acids such as arginine, sodium hydroxymethylglycinate, or combinations thereof.

In some instances, protein hydrolyzates may also be present in body wash compositions according to the disclosure in an amount of up to about 2.0 weight percent, based on the total weight of the body wash composition. Examples of protein hydrolyzates include, but are not limited to, elastin, collagen, keratin, milk protein, soya protein, or wheat protein hydrolyzates, or condensation products thereof with fatty acids, or quaternized protein hydrolyzates, or combinations thereof.

In some instances, body wash compositions according to the disclosure further comprise one or more inorganic electrolyte salts in an amount of from 0 weight percent to about 2 weight percent, based on the total weight of the body wash composition. Such electrolyte salts may be added, for example, to thicken the composition. Inorganic electrolyte salts that can be used are any water-soluble alkali metal, ammonium or alkaline earth metal salt, for example the fluorides, chlorides, bromides, sulfates, phosphates, nitrates or hydrogen carbonates, providing that they are soluble in water at 20° C. in a quantity of at least 1 percent by weight. In some instances, sodium chloride and magnesium chloride are especially preferred.

In some instances, body wash compositions according to the disclosure further comprise additive components, one or more sequestrants (i.e., chelants) such as ethylene diamine tetraacetic acid (EDTA), methylgylcinediactetic acid (MGDA), or phosphonic acids; fragrances; other nonactive ingredients such as panthenol, allantoin, pyrrolidone carboxylic acids or salts thereof; plant extracts; vitamins such as Vitamins A, B, C, or E, or combinations thereof; preservatives such methylchloroisothiazolinone, methylisothiazolinone, paraben, or diazolidinyl urea or combinations thereof; humectants, such as sorbitol, glycerin, propylene glycol, or butylene glycol or combinations thereof; light stabilizers; dyes (or colorants); pearlescers (or opacifiers), such as ethylene glycol monostearate (EGMS) or distearate; antioxidants; viscosifiers (or viscosity builder) such as, for example, alkanol amine, salt, amine oxide; foam boasters including, for example, amino oxide, sarcosinate, or lactylate; foam stabilizers such as, for example, lactylate or gum; active additives such as, for example, zinc pyrithione or salicylic acid; thickeners such as polysaccharides, including for example, xanthan gum, guar gum, agar gum, alginates, tyloses, or celluloses such as carboxymethyl cellulose, or hydroxyethyl cellulose; polyethylene glycol monoesters or diesters of fatty acids having a molecular weight of at least about 200,000; poly(meth)acrylic acid or salts thereof; polyvinyl alcohol; or fatty alcohol ethoxylates; or any combination of any of the foregoing additives. In some instances, preferably, these optional additives are present individually in the composition in an amount of no more than about 2 weight percent and more preferably in an amount of no more than about 0.5 weight percent based on the total weight of the composition.

According to various aspects according to the disclosure, an exemplary method is provided for washing skin using body wash compositions according to the disclosure. Such body wash compositions preferably applied to skin that has first been wetted with water. Following application of the body wash composition to the skin, the composition is typically rinsed from the skin with water to remove any undesirable substances such as dirt or sebum. The body wash compositions described herein have the advantage of cleaning skin.

Body wash compositions according to the disclosure can be formulated utilizing techniques well known to those skilled in the art. The various components that provide the body wash composition according to the disclosure can be added in any order. For example, the amine source can be added to a reactor vessel including at least one surfactant, water and the body wash raw material including the reducing sugar, and thereafter a Maillard reaction inhibiting enzyme can be added to the vessel. In some instances, the addition is preferably a metered addition. In some instances, the addition of the various components that provide the body wash composition according to the disclosure is performed with continuous stirring. In some instances, the addition is preferably performed at room temperature, e.g., from about 20° C. to about 30° C. In some instances, an enzyme enhancing agent can be added during the formulation process to improve the performance of the Maillard reaction inhibiting enzyme. In some instances, the Maillard reaction inhibiting enzyme can be added to a previously formulated body wash composition.

According to various aspects of the disclosure, body wash compositions according to the disclosure are prepared by adding the at least one primary surfactant, optionally a secondary surfactant, an amine source and/or a primary amine source comprising a fermented raw material that includes a reducing sugar, and optionally the sustainable raw material including the reducing sugar to water, then adding the Maillard reaction inhibiting enzyme and thereafter adjusting the pH to about 4 to about 6. The admixture is then allowed to mix for a time period from about 5 minutes to 60 minutes. After this mixing time period, the addition components can be added. If necessary, a final pH adjusting step can be performed to bring the body wash composition to a pH from about 4 to about 6. One skilled in the art will also recognize that there are various other ways to prepare the composition of the present invention.

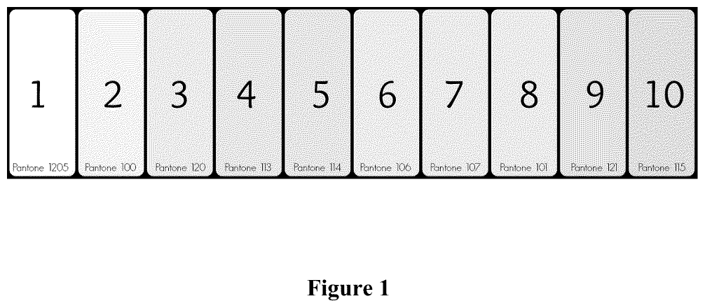

In accordance with various aspects of the disclosure, the yellowing in a body wash composition can be measured through the pantone color systems. FIG. 1 displays the numerical representation of each color of yellow. Table 1 is also the tabular representation of FIG. 1.

TABLE 1

| Numerical Representation | Pantone Number |
|---|---|
| 1 | Pantone 1205 |
| 2 | Pantone 100 |
| 3 | Pantone 120 |
| 4 | Pantone 113 |
| 5 | Pantone 114 |
| 6 | Pantone 106 |
| 7 | Pantone 107 |
| 8 | Pantone 101 |
| 9 | Pantone 121 |
| 10 | Pantone 115 |

Examples have been set forth below for the purpose of further illustrating the present disclosure. The scope according to the disclosure is not limited to any of the examples set forth herein.

### Exemplary Body Wash Composition Formulations

### Prophetic Example 1

Body wash compositions can be prepared by mixing the components in the amounts listed in Table 2. Notably, the body wash compositions can be batched using a standard overhead mixer, target pH 4 to 6. Formula 1 and Formula 2 of Table 2 are substantially identical in composition except that Formula 2 includes a Maillard reaction inhibiting enzyme as described herein, while the Maillard reaction inhibiting enzyme is omitted from Formula 1. Formula 1 is a comparative example (namely CE1), while Formula 2 is an example in accordance with an embodiment according to the disclosure. In Formula 2 below, the Maillard reaction inhibiting enzyme can be added prior to reducing the pH to the target value mentioned above.

TABLE 2

| | Wt % of Formula 1 | Wt % of Formula 2 |
|---|---|---|
| Ingredient | (Comparative) | (Inventive) |
| Water | q/s to 100 | q/s to 100 |
| pH adjuster | As needed to | As needed to |
| (ex. Citric acid) | pH 4 to 6 | pH 4 to 6 |
| Benzalkonium chloride | 0.1 to 1 | 0.1 to 1 |
| (anti-bacterial agent) | | |
| Cocamidopropyl betaine | 1 to 25 | 1 to 25 |
| (primary surfactant) | | |
| Lauramine oxide | 1 to 10 | 1 to 10 |
| (primary surfactant) | | |
| Cetyl trimethyl ammonium | 1 to 10 | 1 to 10 |
| chloride (secondary surfactant) | | |
| Viscosifier | 0.1 to 2 | 0.1 to 2 |
| Thickener | 0.1 to 1 | 0.1 to 1 |
| Biosurfactant | 0.1 to 5 | 0.1 to 5 |
| Maillard reaction inhibiting | 0 | 0.001 to 1 |
| enzyme | | |
| Additives | 0.1 to 2 | 0.1 to 2 |
| Total | 100 | 100 |

### Prophetic Example 2

Body wash compositions can be prepared by mixing the components in the amounts listed in Table 3. Notably, the body wash compositions can be batched using a standard overhead mixer, target pH 4 to 6. Formula 3 and Formula 4 of Table 3 are substantially identical in composition except that Formula 4 includes a Maillard reaction inhibiting enzyme as described herein, while the Maillard reaction inhibiting enzyme is omitted from Formula 3. Formula 3 is a comparative example, while Formula 4 is an example in accordance with an embodiment according to the disclosure. In Formula 4 below, the Maillard reaction inhibiting enzyme can be added prior to reducing the pH to the target value mentioned above.

Formula 5 and Formula 6 of Table 3 are substantially identical in composition except that Formula 6 includes a Maillard reaction inhibiting enzyme as described herein, while the Maillard reaction inhibiting enzyme is omitted from Formula 5. Formula 5 is a comparative example, while Formula 6 is an example in accordance with an embodiment according to the disclosure. In Formula 6 below, the Maillard reaction inhibiting enzyme can be added prior to reducing the pH to the target value mentioned above.

TABLE 3

| | Formula 3 | Formula 4 | Formula 5 | Formula 6 |
|---|---|---|---|---|
| Ingredient | (Comparative) | (Inventive) | (Comparative) | (Inventive) |
| Preservative | 0.01 to 0.6 | 0.01 to 0.6 | 0.01 to 0.6 | 0.01 to 0.6 |
| C12-14 Fatty alcohol ethoxylate | | | 0 to 1 | 0 to 1 |
| (4EO) | | | | |
| Cocamidopropyl Betaine | 1 to 10 | 1 to 10 | 1 to 5 | 1 to 5 |
| Ethylenediamine-N,N′ disuccinic | 0 to 1 | 0 to 1 | 0 to 1 | 0 to 1 |
| acid (sodium salt) | | | | |
| Polyquaternium-7 | 0 to 1 | 0 to 1 | 0 to 1 | 0 to 1 |
| Polyquaternium-10 | | | 0 to 1 | 0 to 1 |
| Non-Aqueous Solvent | 0 to 5 | 0 to 5 | 0 to 5 | 0 to 5 |
| (ex. Glycerin) | | | | |
| C14-16 Olefin sulfonate (sodium | 1 to 10 | 1 to 10 | | |
| salt) | | | | |
| PEG-7 Glyceryl Cocoate | 0 to 1 | 0 to 1 | | |
| PEG-150 Pentaerythrityl | 0 to 0.5 | 0 to 0.5 | | |
| Tetrastearate | | | | |
| PPG-2 Hydroxyethyl Cocamide | 0 to 0.5 | 0 to 0.5 | | |
| Sodium Chloride | 0 to 1 | 0 to 1 | 0 to 1 | 0 to 1 |
| Beneficial Additive (ex. Vitamin | 0 to 1 | 0 to 1 | 0 to 1 | 0 to 1 |
| E) | | | | |
| Fragrance/Dye | 0 to 3 | 0 to 3 | 0 to 3 | 0 to 3 |
| PEG-120 Methyl Glucose | | | 0 to 1 | 0 to 1 |
| Trioleate | | | | |
| Biopolymer (starch-based) | 0 to 4 | 0 to 4 | 0 to 4 | 0 to 4 |
| Biosurfactant (fermented from | 0 to 8 | 0 to 8 | 0 to 8 | 0 to 8 |
| glucose or fructose) | | | | |
| Sustainable Solvent | 0 to 4 | 0 to 4 | 0 to 4 | 0 to 4 |
| pH Adjuster | N/A | As needed | N/A | N/A |
| (ex. Citric Acid) | | to pH 7.6 | | |
| Maillard reaction inhibiting | 0 | 0.001 to 1 | 0 | 0.001 to 1 |
| enzyme | | | | |
| pH Adjuster | N/A | As needed | N/A | As needed |
| | | to pH 4 to 6 | | to pH 4 to 6 |
| Water | q/s to 100 | q/s to 100 | q/s to 100 | q/s to 100 |

Formulas 7 and 8 were created by mixing the above components together using a standard overhead mixer at room temperature for 1 hour. Glucose was added to illustrate the effects of a reducing sugar. The formulations of formulas 7 and 8 are seen in Table 4 below.

TABLE 5

| | | Formula 7 | Formula 8 |
|---|---|---|---|
| | | (Comparative, | (Inventive, |
| Ingredient | Active % | wt %) | wt %) |
| Water | 100 | 57.69 | 57.79 |
| Glucose | 100 | 0.50 | 0.50 |
| Hexose oxidase | 100 | 0.00 | 0.10 |
| Benzalkonium Chloride | 50 | 0.26 | 0.26 |
| Cocoamidopropyl betaine | 40 | 20.00 | 20.00 |
| Amine oxide | 30 | 7.00 | 7.00 |
| Cetyl Trimethyl Ammonium | 30 | 5.00 | 5.00 |
| Chloride | | | |
| PEG-150 Pentaerythrityl | 25 | 1.00 | 1.00 |
| Tetrastearate and PPG-2 | | | |
| hydroxyethyl cocamide | | | |
| EDTA | 40 | 0.05 | 0.05 |
| Na-benzoate | 100 | 0.40 | 0.40 |
| Monoethanol amine (MEA) | 100 | 3.00 | 3.00 |
| Citric Acid (pH adjust to 5.5, | 100 | 5.00 | 5.00 |
| then QS) | | | |
| Total | | 100.00 | 100.00 |

Formulas 7 and 8 were tested for yellowing with the results displayed below in Table 6. Formula 8, with the addition of the Maillard reaction inhibiting enzyme, prevented yellowing in the body wash composition.

TABLE 6

| Formula # (Storage Conditions) | Pantone Color ranking (0-10)* |
|---|---|
| Formula 7 (1-week at 24 C.) | 0 |
| Formula 8 (1-week at 24 C.) | 0 |
| Formula 7 (1-week at 52 C.) | 6 |
| Formula 8 (1-week at 52 C.) | 4 |

Formulas 9 and 10 were created by mixing the above components together using a standard overhead mixer at room temperature for 1 hour. Glucose was added to illustrate the effects of a reducing sugar. The formulations of formulas 9 and 10 are seen in Table 7 below.

TABLE 7

| | | Formula 9 | Formula 10 |
|---|---|---|---|
| Ingredient | Active % | (Comparative, wt %) | (Inventive, wt %) |
| Water | 100 | 58.63 | 58.73 |
| Glucose | 100 | 0.50 | 0.50 |
| Hexose oxidase | 100 | 0.00 | 0.10 |
| Trisodium | 30 | 0.05 | 0.05 |
| Ethylenediamine | | | |
| Disuccinate | | | |
| Polyquaternium-7 | 9.3 | 2.00 | 2.00 |
| Glycerin | 100 | 2.00 | 2.00 |
| Sodium C14-16 Olefin | 38 | 12.00 | 12.00 |
| Sulfonate | | | |
| Cocoamidopropyl | 40 | 16.00 | 16.00 |
| betaine | | | |
| Na-benzoate | 100 | 0.40 | 0.40 |
| PEG-7 Glycerol | 100 | 0.20 | 0.20 |
| Cocoate | | | |
| Vitamin E acetate | 100 | 0.02 | 0.02 |
| PEG-150 | 25 | 0.10 | 0.10 |
| Pentaerythrityl | | | |
| Tetrastearate and | | | |
| PPG-2 Hydroxyethyl | | | |
| Cocamide | | | |
| Monoethanol amine | 100 | 3.00 | 3.00 |
| (MEA) | | | |
| Citric acid (pH adjust | 50 | 5.00 | 5.00 |
| to 5.5, then QS) | | | |
| Total | | 100.00 | 100.00 |

Formulas 9 and 10 were tested for yellowing with the results displayed below in Table 8. Formula 10, with the addition of the Maillard reaction inhibiting enzyme, prevented yellowing in the body wash composition.

TABLE 8

| Formula # (Storage Conditions) | Pantone Color ranking (0-10) |
|---|---|
| Formula 9 (1-week at 24 C.) | 1 |
| Formula 10 (1-week at 24 C.) | 0 |
| Formula 9 (1-week at 52 C.) | 6 |
| Formula 10 (1-week at 52 C.) | 4 |

Formulas 11 and 12 were created by mixing the above components together using a standard overhead mixer at room temperature for 1 hour. Glucose was added to illustrate the effects of a reducing sugar. The formulations of formulas 11 and 12 are seen in Table 9 below.

TABLE 9

| | | Formula 11 | Formula 12 |
|---|---|---|---|
| Ingredient | Active % | (Comparative, wt %) | (Inventive, wt %) |
| Water | 100 | 55.01 | 55.11 |
| Glucose | 100 | 0.50 | 0.50 |
| Hexose oxidase | 100 | 0.00 | 0.10 |
| Polyquaternium-10 | 30 | 0.40 | 0.40 |
| EDTA | 40 | 0.05 | 0.05 |
| Glycerin | 100 | 1.30 | 1.30 |
| Cocoamidopropyl | 40 | 9.23 | 9.23 |
| betaine | | | |
| Sodium Lauryl Sulfate | 29 | 23.75 | 23.75 |
| Polyquaternium-7 | 9.3 | 1.00 | 1.00 |
| PEG-120 Methyl | 100 | 0.25 | 0.25 |
| Glucose Trioleate, | | | |
| Propanediol | | | |
| Na-benzoate | 100 | 0.40 | 0.40 |
| Sodium chloride fine- | 100 | 0.01 | 0.01 |
| medium | | | |
| Monoethanol amine | 100 | 3.00 | 3.00 |
| (MEA) | | | |
| Citric acid (pH adjust | 50 | 5.00 | 5.00 |
| to 5.5, then QS) | | | |
| Total | | 100.00 | 100.00 |

Formulas 11 and 12 were tested for yellowing with the results displayed below in Table 10. Formula 12, with the addition of the Maillard reaction inhibiting enzyme, prevented yellowing in the body wash composition.

TABLE 10

| Formula # (Storage Conditions) | Pantone Color ranking (0-10) |
|---|---|
| Formula 11 (1-week at 24 C.) | 1 |
| Formula 12 (1-week at 24 C.) | 0 |
| Formula 11 (1-week at 52 C.) | 5 |
| Formula 12 (1-week at 52 C.) | 3 |

While the present disclosure has been particularly shown and described with respect to preferred embodiments thereof, it will be understood by those skilled in the art that the foregoing and other changes in forms and details may be made without departing from the spirit and scope according to the disclosure. It is therefore intended that the present disclosure not be limited to the exact forms and details described and illustrated, but fall within the scope of the appended claims.

1. A body wash composition comprising:
at least one surfactant;
an amine source;
a sustainable raw material including a reducing sugar;
a Maillard reaction inhibiting enzyme that inhibits reaction between the amine source and the reducing sugar; and
at least 40 weight percent water, based on the total weight of the body wash composition.

2. The body wash composition of claim 1, wherein the at least one surfactant comprises a primary surfactant, where the primary surfactant is present in an amount from about 1 weight percent to about 40 weight percent, based on the total weight of the body wash composition.

3. The body wash composition of claim 2, wherein the at least one surfactant further comprises a secondary surfactant, where the secondary surfactant is present in an amount from about 0.1 weight percent to about 15 weight percent, based on the total weight of the body wash composition.

4. The body wash composition of claim 1, wherein the amine source comprises a protein, an amine containing surfactant, a chelating agent, an amine containing enzyme, or a hydrolyzed protein, or any combination thereof.

5. The body wash composition of claim 1, wherein the amine source is present in an amount from about 0.05 weight percent to about 30 weight percent, based on the total weight of the body wash composition.

6. The body wash composition of claim 1, wherein the sustainable raw material comprises a sustainable polymer or a sustainable solvent, or a combination of a sustainable polymer and a sustainable solvent.

7. The body wash composition of claim 6, wherein the sustainable solvent comprises sorbitol, xylitol, mannitol, lactitol, isomalt, maltitol, a hydrogenated starch hydrolysate, sucrose, stachyose, verbascose, trehalose, or raffinose, or any combination thereof.

8. The body wash composition of claim 6, wherein the sustainable polymer comprises a starch, a glycogen, a galactogen, a cellulose, or a chitin, or any combination thereof.

9. The body wash composition of claim 1, wherein the Maillard reaction inhibiting enzyme comprises hexose oxidase, fructosamine oxidase, fructosamine kinase, or carbohydrate oxidase, or any combination thereof.

10. The body wash composition of claim 1, wherein the Maillard reaction inhibiting enzyme is present in an amount of from about 0.001 weight percent to about 3 weight percent, based on the total weight of the composition.

11. A body wash composition comprising:
at least one surfactant;
a primary amine source comprising a fermented raw material that includes a reducing sugar;
a Maillard reaction inhibiting enzyme that inhibits reaction between the primary amine source and the reducing sugar; and
at least 40 weight percent water, based on the total weight of the composition.

12. The body wash composition of claim 11, wherein the at least one surfactant comprises a primary surfactant, wherein the primary surfactant is present in an amount from about 1 weight percent to about 40 weight percent, based on the total weight of the body wash composition.

13. The body wash composition of claim 12, wherein the at least one surfactant further comprises a secondary surfactant, wherein the secondary surfactant is present in an amount from about 0.1 weight percent to about 15 weight percent, based on the total weight of the body wash composition.

14. The body wash composition of claim 11, wherein the primary amine source comprises a biosurfactant.

15. The body wash composition of claim 11, wherein the primary amine source is present in an amount ranging from about 0.05 weight percent to about 30 weight percent, based on the total weight of the body wash composition.

16. The body wash composition of claim 11, wherein the Maillard reaction inhibiting enzyme comprises fructosamine oxidase, fructosamine kinase, or carbohydrate oxidase, or any combination thereof.

17. The body wash composition of claim 11, wherein the Maillard reaction inhibiting enzyme is present in an amount ranging from about 0.001 weight percent to about 3 weight percent, based on the total weight of the body wash composition.

18. The body wash composition of claim 11, further comprising a sustainable solvent including a reducing sugar or a sustainable polymer including a reducing sugar, or a combination of a sustainable solvent including a reducing sugar and a sustainable polymer including a reducing sugar.

19. The body wash composition of claim 18, wherein the sustainable solvent comprises sorbitol, xylitol, mannitol, lactitol, isomalt, maltitol, a hydrogenated starch hydrolysate, sucrose, stachyose, verbascose, trehalose, or raffinose, or any combination thereof.

20. The body wash composition of claim 18, the sustainable polymer comprises a starch, a glycogen, a galactogen, a cellulose, or a chitin, or any combination thereof.
